(19) United States
(12) Patent Application Publication
(10) Pub. No.: US 2023/0091503 A1
(43) Pub. Date: Mar. 23, 2023

(54) PREPARATION METHOD FOR MICRONEEDLE PATCH

(71) Applicant: East China University of Science and Technology, Shanghai (CN)

(72) Inventors: Changsheng Liu, Shanghai (CN); Hongyan He, Shanghai (CN); Lili Jin, Shanghai (CN); Dian Zhang, Shanghai (CN)

(21) Appl. No.: 17/990,038

(22) Filed: Nov. 18, 2022

Related U.S. Application Data
(63) Continuation of application No. PCT/CN2021/113007, filed on Aug. 17, 2021.

(30) Foreign Application Priority Data
Dec. 29, 2020 (CN) 202011598079.4

(51) Int. Cl. A61M 37/00; B29C 33/38

(52) U.S. Cl. CPC A61M 37/0015; B29C 33/3842; A61M 2037/0053; A61M 2037/0023; A61M 2037/0046

(57) ABSTRACT

The present invention relates to a preparation method for a microneedle patch. Specifically, the present invention provides a preparation method for a microneedle patch, and the method comprises steps: (1) milling a base into a master mold of the microneedle patch; (2) conducting surface treatment on the master mold, to obtain the treated master mold; (3) conducting reverse molding on a surface of the treated master mold, conducting deaeration, curing, and demolding, to obtain the cured daughter mold; and (4) casting the modification solution on the surface of the daughter mold, and then conducting deaeration, drying and curing, to obtain the microneedle patch. The method for preparing the microneedle patch in the present invention can greatly reduce the milling difficulty, and save the milling cost and time. The microneedle molds with different aspect ratios, areas and shapes can also be prepared as required, following with reverse molding to diverse microneedle patches. Therefore, a feasible implementation scheme is provided in present invention for the production application of the microneedles and the feasibility of the wide application is greatly enhanced.

### TECHNICAL FIELD

The present invention relates to the technical field of micro-nanometers, and specifically, the present invention provides a preparation method for a microneedle patch.

### BACKGROUND

The microneedle patch is a patch of micron-scale needle array that can pierce the epidermis painfully or even painlessly to deliver drugs into the dermis. Compared to the subcutaneous injection, patients prefer painlessly transdermal drug delivery of microneedles, especially for patients with frequent drug injections, such as insulin or hormone therapy. Therefore, the microneedles have a broad application market. Compared to the dressing administration, the microneedle administration can deliver the drugs to the dermis fast by physically generating transdermal channels. Besides, the molecular weight of drugs loaded in microneedles is not limited, so proteins and other macromolecular drugs can be delivered unhinderedly.

The geometry parameter of microneedles is a key issue for the preparation and application. The size and shape of microneedles determine the penetration ability, drug loading capacity and drug release rate thereof. It is an eagle issue to fabricate microneedle patches with specific types for different requirements. The typical methods for preparing microneedle molds or microneedles are stereographic photoetching, dry or wet etching, two-photon polymerization, micro-milling, etc. Compared to other methods, the micro-milling method can accurately control the size and be capable of mass production. Consequently, the micro-milling has great potential in practical application. However, the micro-milling method needs expensive instruments with high-strength milling tools, so the excessive cost makes it unsuitable for large-scale commercial promotion. In addition, although a simple droplet-born air blowing approach mentioned in the prior art can prepare the microneedles rapidly, the consistency of each needle in one patch is poor and the microneedle tip is easy to draw. The microneedles produced in each batch cannot be guaranteed to be uniform in specification. Therefore, the droplet-born air blowing approach is also not suitable for practical production application.

At present, the materials used for manufacturing the microneedle mold or the microneedle patch are silica, metal, glass, and polymer. Metal and the silica materials are commonly used in scientific research, which are subject to the practical application with the limitation of tedious preparation time, difficult preparation method and high preparation cost. Moreover, the metal microneedles are easy to cause adverse reactions such as inflammation, skin ulceration of using positions.

Consequently, how to prepare microneedle patches or molds quickly, obtain biocompatible microneedle arrays with optional types and adjustable size/areas, and reduce the cost and the preparation difficulty degree at the same time are still urgent issues to be solved for the practical application of the microneedles at present.

### SUMMARY

The purpose of the present invention is to provide a method for simply and quickly preparing a microneedle patch. The method overcomes the disadvantages of low designability, complex preparation process, expensive instrument and tedious preparation time for the microneedles. Microneedle molds with different shapes, different size and different areas can be prepared as required. The master mold can be quickly and completely reversed into a microneedle patch. Therefore, the cost and difficulty of microneedle preparation are reduced and the preparation time is saved for the commercial process.

In a first aspect, the present invention provides a preparation method for a microneedle patch, and the method comprises the steps of:

(1) milling a base into a master mold of the microneedle patch;

(2) conducting surface treatment on the master mold, to obtain the treated master mold;

(3) conducting reverse molding on a surface of the treated master mold, including deaeration, curing, and demolding, to obtain the cured daughter mold; and

(4) casting the modification solution on the surfaces of the daughter mold, and then deaeration, drying and curing, to obtain the microneedle patch.

In another preferred example, the shape of the microneedles is designed by 3Ds MAX mapping software.

In another preferred example, in the step (1), the milling is performed by using a milling machine.

In another preferred example, in the step (1), the milling machine is a desktop type automatic milling machine.

In another preferred example, the milling machine is an MDX-50 desktop type automatic milling machine (Roland, Japan).

In another preferred example, the milling machine has the accuracy of 0.005-0.015 mm.

In another preferred example, in the step (1), the shape of the microneedle is conical, pyramidal, cross-conical, cylindrical-conical combined shape, or a combination thereof.

In another preferred example, in the step (1), the aspect ratio of the microneedles is (1-3):1, for example, 1:1, 2:1 or 3:1.

In another preferred example, the height of the microneedle is 800-2200 μm, preferably 900-1100 μm.

In another preferred example, in the step (1), the master mold contains multiple microneedles.

In another preferred example, the distance between central axes of adjacent microneedles is 800-2200 μm, preferably 900-1100 μm.

In another preferred example, the bottom diameter of the conical microneedle is 250-1100 μm, preferably 450-550 μm.

In another preferred example, the bottom side length of the pyramidal microneedle is 500 μm.

In another preferred example, the bottom diameter of the cross-conical microneedle is 450-550 μm, and the thickness of each side is 90-110 μm.

In another preferred example, a cylindrical-conical combined microneedle is composed of a cylindrical base and a conical tip. The cylindrical base and the conical tip are coaxial. The bottom diameter is 450-550 μm, and the height of the cylindrical base and the conical tip is 450-550 μm.

In another preferred example, in the step (1), the material of the base is selected from the following groups: polymer-based composite, gypsum, modelling wax, or a combination thereof.

In another preferred example, the polymer-based composite is selected from the following groups: polyurethane-based resin material, ABS resin, acrylics, polyacetal-based composite material, polycarbonate-based composite material, or a combination thereof.

In another preferred example, the microneedles on the master mold are arranged in an array.

In another preferred example, in the step (2), the surface treatment is gold-sputtering treatment, and the sputtering treatment conditions are: the current is 5-20 mA, and the sputtering time is 0.5-1.5 min.

In another preferred example, the current is 10-18 mA.

In another preferred example, the sputtering time is 0.5-1.5 min.

In another preferred example, in the step (2), the surface treatment is titanium-, aluminium- or platinum-sputtering treatment, and the thickness is less than 10 nm.

In another preferred example, in the step (2), the surface treatment is plasma treatment. The treatment gas is oxygen, argon or ammonia, the treatment time is 5-30 min, preferably 10-20 min, and the treatment power is 60-80 W.

In another preferred example, in the step (2), the surface treatment is the spraying of the drying release agent.

In another preferred example, in the step (3), the reverse molding is conducted on the surface of the treated master mold.

In another preferred example, the reverse molding is conducted by the mixture containing the dimethyl silicone and the curing agent (Dow Corning SYLGARD 184 kit).

In another preferred example, the weight ratio of the dimethyl silicone to curing agent is 8-12:1.

In another preferred example, the weight ratio of dimethyl silicone to curing agent is 10:(0.8-1.5).

In another preferred example, in the step (3), the deaeration needs to be conducted by negative pressure before curing.

In another preferred example, in the step (3), the deaeration needs to be conducted by high-speed centrifugation at 4° C. before curing.

In another preferred example, in the step (3), the curing temperature is 85-95° C.

In another preferred example, in the step (3), the curing time is 0.5-1.5 h.

In another preferred example, in the step (4), the modification solution is used for modifying the surface of the daughter mold.

In another preferred example, the modification solution is PVA aqueous solution with the weight fraction of 2-20 w.t.%, preferably 6-10 w.t.%.

In another preferred example, the modification solution is selected from the following groups: solution of chitosan or a derivative thereof, solution of alginate or derivatives thereof, solution of hyaluronic acid or derivatives thereof, collagen solution, silk fibroin solution, carboxymethylcellulose solution, dextrose solution, chondroitin sulfate solution, lactic acid-hydroxyl glycolic acid copolymer solution, polycaprolactone solution, polylactic acid solution, polyethylene glycol solution, polyvinylpyrrolidone solution, or a combination thereof.

In another preferred example, in the step (4), the deaeration needs to be conducted by negative pressure before curing.

In another preferred example, in the step (4), the deaeration needs to be conducted by high-speed centrifugation at normal temperature before curing.

In another preferred example, in the step (4), the drying temperature is 20° C.-70° C., preferably 40-60° C., and more preferably 50° C.

In another preferred example, in the step (4), the drying time is 10-48 h, preferably 15-30 h, and more preferably 22-26 h.

In the second aspect, the present invention provides a microneedle patch, which is prepared by the above method of the present invention.

It should be understood that each of the above technical characteristics of the present invention and each of the technical characteristics specifically described in the following (e.g., embodiments) may be combined with each other with the scope of the present invention, thereby constituting a new or preferred technical solution. Due to space limitations, it will not be repeated herein.

### DESCRIPTION OF DRAWINGS

FIG. 1 is a flow chart of the preparation for a microneedle array, wherein (a) is a mold milling process, (b) is a polyurethane-based master mold obtained after milling, (c) is a PDMS daughter mold, and (d) is a PVA microneedle patch.

FIG. 2 is a SEM picture of microneedle arrays with different shapes prepared in embodiment 1, and the aspect ratio of the microneedles is 2:1, wherein (2a) is a conical microneedle array, (2b) is a pyramidal microneedle array, (2c) is a cross-conical microneedle, and (2d) is a cylindrical-conical combined microneedle. The scale bar is 500 μm.

FIG. 3 is a SEM picture of conical microneedles with different proportions prepared in the embodiment, wherein in (3a), the aspect ratio of each microneedle is 1:1, in (3b), the aspect ratio of each microneedle is 2:1, in (3c), the aspect ratio of each microneedle is 2.5:1, and in (3d), the aspect ratio of each microneedle is 3:1. The scale is 500 μm.

FIG. 4 is a picture of conical microneedle arrays with different areas, and the aspect ratio of the microneedles is 3:1, wherein (4a) is a microneedle array with the area of (35×35) mm², and (4b) is a microneedle array with the area of (6×6) mm².

FIG. 5 is a comparison between PDMS daughter molds obtained after reverse molding of the master molds through surface treatment or without treatment, wherein (5a) is a master mold after sputtering treatment and reverse molding of embodiment 1, (5b) is a master mold after reverse molding without sputtering treatment of reference example 1, (5c) is a PDMS daughter mold obtained by reverse molding of the master mold after sputtering treatment of embodiment 1, and (5d) is a PDMS daughter mold obtained by reverse molding of the master mold without sputtering treatment of reference example 1.

### DETAILED DESCRIPTION

The present invention has developed a preparation method of microneedle patches for extensive and in-depth research. The method overcomes the disadvantages of low designability, complex preparation process, expensive instrument and tedious preparation time for preparing microneedle patches. Microneedle molds with different shapes, different size and different areas can be prepared as required. The master mold can be quickly and completely reversed into a microneedle patch. Therefore, the cost and difficulty of microneedle preparation are reduced and the preparation time is saved, which provide a new method for commercialized large-scale application of a microneedle patch.

### Terms

Unless otherwise defined, all technical and scientific terms used herein have the same meanings as those generally understood by those skilled in the art in the present invention.

The terms “comprise”, “include” and “contain” used herein can be used interchangeably, and not only comprise open definition, and but also comprise semi-enclosed and closed definitions. In other words, the terms comprise “constitute” and “basically constitute”.

### Preparation Method for Microneedle Patch

The present invention provides a preparation method for a microneedle patch, and the method comprises steps (1)-(4), specifically as follows:

step (1) milling a base into a master mold of the microneedle patch;

In another preferred example of the present invention, in the step (1), the milling is performed by using a milling machine.

Preferably, the milling machine is a desktop type automatic milling machine. For example, the milling machine is an MDX-50 desktop type automatic milling machine (Roland, Japan).

In another preferred example, the milling machine has the accuracy of 0.005-0.015 mm.

In another preferred example of the present invention, the shapes of the microneedles are selected from the following groups: conical, pyramid, cross-conical, cylindrical-conical combined shape, or a combination thereof.

In another preferred example of the present invention, the aspect ratio of the microneedles is (1-3):1, for example, 1:1, 2:1 or 3:1.

In another preferred example, the height of the microneedle is 800-2200 μm, preferably 900-1100 μm.

In another preferred example, the master mold contains multiple microneedles.

In another preferred example, the distance between central axes of adjacent microneedles is 800-2200 μm, preferably 900-1100 μm.

In another preferred example, the bottom diameter of the conical microneedle is 250-1100 μm, preferably 450-550 μm.

In another preferred example, the bottom side length of the pyramid microneedle is 500 μm.

In another preferred example, the bottom diameter of the cross-conical microneedle is 450-550 μm, and the thickness of each side is 90-110 μm.

In another preferred example, a cylindrical-conical combined microneedle is composed of a cylindrical base and a conical tip. The cylindrical base and the conical tip are coaxial. The bottom diameter is 450-550 μm, and the height of the cylindrical base and the conical tip is 450-550 μm.

In another preferred example, the material of the base is selected from polymer-based composite, gypsum, modelling wax, or a combination thereof.

In another preferred example, the polymer-based composite is selected from the following groups: polyurethane-based resin material, ABS resin, acrylics, polyacetal-based composite material, polycarbonate-based composite material, or a combination thereof.

In another preferred example, the microneedles on the master mold are arranged in an array.

Step (2): conducting the surface treatment on the master mold, to obtain the treated master mold;

In one preferred example of the present invention, the surface treatment is sputtering treatment, and the sputtering treatment conditions are: the current is 5-20 mA, and the sputtering time is 0.5-1.5 min;

In another preferred example, the current is 10-18 mA.

In another preferred example, the sputtering time is 0.5-1.5 min.

In another preferred example, the surface treatment is titanium-, aluminium- or platinum-sputtering treatment, and the thickness is not more than 10 nm.

In another preferred example, the surface treatment is plasma treatment. The treatment gas is oxygen, argon or ammonia, the treatment time is 5-30 min, preferably 10-20 min, and the treatment power is 60-80 W.

In another preferred example, the surface treatment is the spraying of the drying release agent.

Step (3): conducting reverse molding on a surface of the treated master mold, including deaeration, curing and demolding, to obtain the cured daughter mold;

In one preferred example of the present invention, in the step (3), the reverse molding is conducted on the surface of the treated master mold.

In another preferred example, the reverse molding is conducted by the mixture containing the dimethyl silicone and the curing agent (Dow Corning SYLGARD 184).

In another preferred example, the weight ratio of the dimethyl silicone to curing agent is 8-12:1.

In another preferred example, the weight ratio of the dimethyl silicone to curing agent is 10:(0.8-1.5).

In another preferred example, in the step (3), the deaeration needs to be conducted by negative pressure before curing.

In another preferred example, in the step (3), the deaeration needs to be conducted by high-speed centrifugation at 4° C. before curing.

In another preferred example, in the step (3), the curing temperature is 85-95° C.

In another preferred example, in the step (3), the curing time is 0.5-1.5 h.

Step (4): casting the modification solution on the surface of the daughter mold, and then drying and curing, to obtain the microneedle patch;

In another preferred example, the modification solution is used for modifying the surface of the daughter mold.

In another preferred example, the modification solution is PVA aqueous solution with the weight fraction of 2-20 w.t.%, preferably 6-10 w.t.%.

In another preferred example, the modification solution is selected from the following groups: solution of chitosan or a derivative thereof, solution of alginate or derivatives thereof, solution of hyaluronic acid or derivatives thereof, collagen solution, silk fibroin solution, carboxymethylcellulose solution, dextrose solution, chondroitin sulfate solution, lactic acid-hydroxyl glycolic acid copolymer solution, polycaprolactone solution, polylactic acid solution, polyethylene glycol solution, polyvinylpyrrolidone solution, or a combination thereof.

In another preferred example, in the step (4), the deaeration needs to be conducted by negative pressure before curing.

In another preferred example, in the step (4), the deaeration needs to be conducted by high-speed centrifugation at normal temperature before curing.

In another preferred example, in the step (4), the drying temperature is 20° C.-70° C., preferably 40-60° C., and more preferably 50° C.

In another preferred example, in the step (4), the drying time is 10-48 h, preferably 15-30 h, and more preferably 22-26 h.

The present invention comprises the major advantages:

the present invention can prepare microneedles with different types by micro-milling.

Firstly, different from the preparation of microneedle molds or microneedles in other inventions, which takes metal material as the base material for milling, the present invention takes resin matrix composite material as the base material, saving the cost and reducing the strength requirements for micro-milling instruments and milling tools. The instruments that can machine the metal-based materials need to be equipped with cooling water and circulating water systems, cooperating with high cutting power and high-strength cutting tools, to ensure the regular rotation of the spindle and normal service conditions of cutting tools during process. However, the micro-milling instruments for milling the resin-based material adopted in the present invention do not need to be equipped with the cooling water and circulating water system. At the same time, the strength of the resin-based material is lower than that of the metal-based material or silica-based material, so that the milling instruments do not require excessive power. In addition, the milling tools used for cutting the metal- or the silica-based materials are easy to wear. However, the repeatability of tools used for cutting the resin-based materials is high with low wear rate. In addition, the micro-milling instruments used in the present invention have strong compatibility, which can recognize various formats of 3D graphics with low requirements for designing software. At the same time, instrument used in present invention possesses its own software for design. Therefore, in terms of milling instrument, the present invention can greatly reduce the milling difficulty and cost.

Compared with the silica-, metal- or the glass-based materials, the resin-based composites in the present invention are easy to obtain with cheap price, so the preparation cost is obviously reduced. Besides, the resin is easy to shape without breaking and can be repeatedly moulded. The number of times of repeated milling can be reduced. Therefore, in terms of the milled materials, the present invention can not only reduce the cost of mold, but also reduce the milling times to save the preparation time of the microneedle patch.

The present invention can prepare varied specifications of high-precision microneedle molds and patches. The aspect ratio is a focus for microneedles preparation and application. The microneedles are easy to pierce the skin with large aspect ratio. However, the difficulty of preparation also raises with the increased aspect ratio. The practical microneedle patches are usually prepared by a template method or directly machined, wherein the template method can be used many times to reduce the cost for preparing microneedle patches. However, the microneedles prepared by the template method are easy to be brittle in the demolding process, so the aspect ratio of microneedles is generally not more than 2:1. The ultra-long microneedles introduced in previous inventions or literatures need the extra fine wire to be arranged neatly as a microneedle template. Nevertheless, this method does not explain the preparation process of the extra fine wire, and cannot guarantee that every arrangement of the wire is consistent.

Thus, the repeatability is poor. The microneedle mold with an aspect ratio of 3:1 can be easily and quickly prepared by the micro-milling instrument in the present invention. The brittle fracture of a needle tip will not occur in the process of the reverse molding with maintaining well shape and aspect ratio. In addition, the present invention can prepare a large area of microneedle patch with the array of 35×35 (area of 35×35 mm²), keeping an aspect ratio of 3:1, which is not limited to research and application, but provides the possibility for subsequent commercial promotion. Finally, the present invention can manufacture microneedles with different shapes (such as conical, pyramidal, cross-conical, conical-conical combined shape) designed by the software such as 3Ds Max, and also can integrate varied shapes on the same patch as required, which hold a high freedom degree of design. Therefore, in terms of the milling scope, the present invention can not only overcome the limitation of the aspect ratio in the traditional milling, but also prepare single or combined microneedles with different areas and different shapes.

Therefore, the present invention can greatly reduce the milling difficulty, and save the milling cost and time. The microneedle molds with different aspect ratios, areas and shapes can also be prepared as required, following with reverse molding to diverse microneedle patches. Therefore, a feasible implementation scheme is provided in present invention for the production application of the microneedles and the feasibility of the wide application is greatly enhanced.

The present invention is further described below in combination with the specific embodiments. It should be understood that the embodiments are only used for illustrating the present invention and limiting the scope of the present invention. Experimental methods without specific conditions are carried out usually under conventional conditions or the conditions recommended by the manufacturer. Unless otherwise specified, the percentages and parts below are based on weight

### Embodiment 1

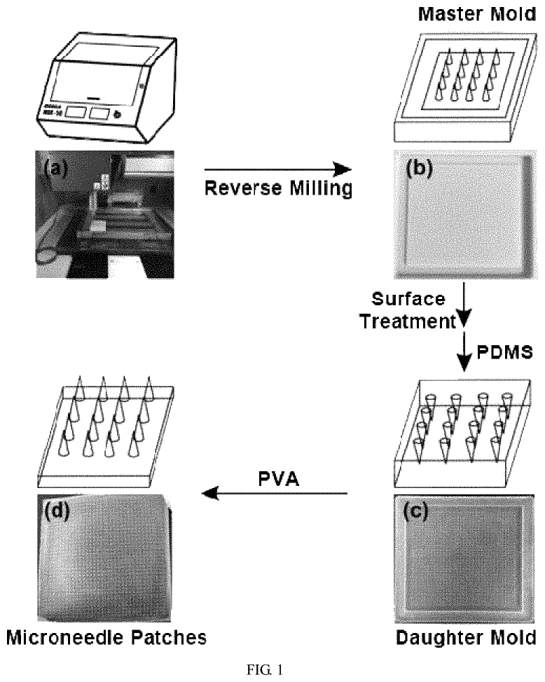

Embodiment 1 provides a preparation method of microneedle patches, a method route thereof refers to FIG. 1, and the method comprises the steps of:

(1) designing conical, pyramidal, cross-conical, cylindrical-conical combined microneedle arrays by adopting a 3Ds MAX mapping software;

(2) according to the software design of step (1), milling a surface of the polyurethane-based resin material by using the MDX-5 desktop type automatic milling machine (Roland, Japan), designing milling cutter operation modes, and then conducting milling, to obtain conical, pyramidal, cross-conical, cylindrical and conical composite microneedle master molds with the aspect ratio of 2:1. The height of the conical, pyramidal, cross-conical, cylindrical-conical combined microneedles is 1000 μm, and the distance between central axes of adjacent microneedles is 1000 μm. The bottom diameter of the conical microneedle is 500 μm. The bottom side length of the pyramidal microneedle is 500 μm. The bottom diameter of the cross-conical microneedle is 500 μm, and the thickness of each side is 100 μm. The combined microneedle is composed of a cylindrical base and a conical tip. The cylindrical base and the conical tip are coaxial. The bottom diameter is 500 μm, and the height of the cylindrical base and the conical tip is 500 μm.

(3) conducting surface sputtering treatment on a master mold by gold-sputtering instrument (SAINTINS, JS-1600), wherein the sputtering time is 1 min, and the current is 14 mA;

(4) casting the mixture (Dow Corning SYLGARD 184) containing the dimethyl silicone (PDMS) and the curing agent with the weight ratio of 10:1 on a surface of the master mold after surface sputtering treatment, removing bubbles from the mixture by negative pressure, placing in an oven for 1 h at 90° C., and then curing and demolding to obtain PDMS daughter mold; and

(5) casting 8 w.t.% of PVA aqueous solution on the surface of the PDMS daughter mold, following with deaeration by negative pressure, and then placing in the oven at 50° C. and drying for 24 h, to obtain a PVA microneedle array, thereby obtaining conical, pyramidal, cross-conical, cylindrical-conical combined microneedle patches.

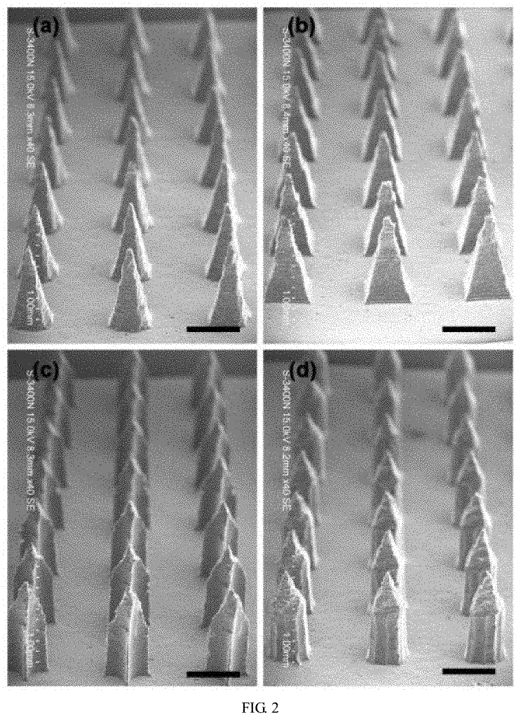

The microneedle array diagrams of conical, pyramidal, cross-conical, cylindrical-conical combined microneedles prepared in embodiment 1 are shown in FIG. 2.

### Embodiment 2

Embodiment 2 is almost the same as Embodiment 1, and the difference is only that:

in the step (2), a conical microneedle master mold at the aspect ratio of 1:1 is obtained by the MDX-50 desktop type automatic milling machine. The height of the conical microneedle is 1 mm, the bottom diameter is 1 mm, and the distance between center axle of adjacent conical microneedles is 2 mm; and

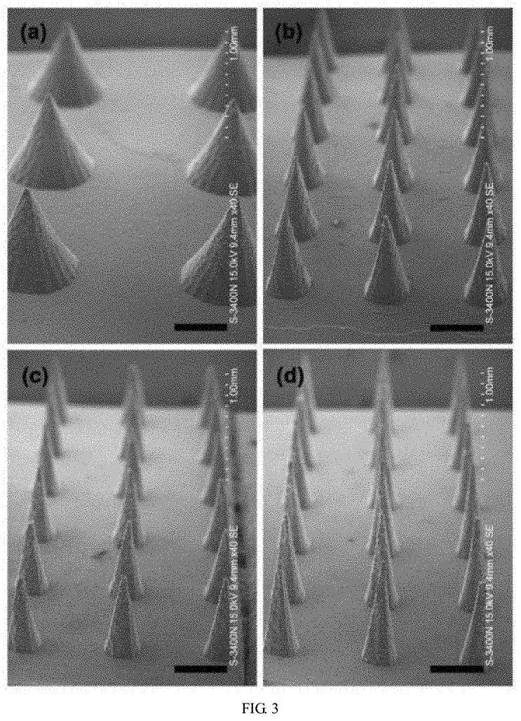

the conical microneedle array diagram prepared in embodiment 2 is shown in FIG. 3a.

### Embodiment 3

Embodiment 3 is almost the same as Embodiment 1, and the difference is only that:

in the step (2), a conical microneedle master mold at the aspect ratio of 2:1 is obtained by the MDX-50 desktop type automatic milling machine. The height of the conical microneedle is 1 mm, the bottom diameter is 0.5 mm, and the distance between center axle of adjacent conical microneedles is 1 mm; and

the conical microneedle array diagram prepared in embodiment 3 is shown in FIG. 3b.

### Embodiment 4

Embodiment 4 is almost the same as Embodiment 1, and the difference is only that:

in the step (2), a conical microneedle master mold at the aspect ratio of 2.5:1 is obtained by the MDX-50 desktop type automatic milling machine. The height of the conical microneedle is 1 mm, the bottom diameter is 0.4 mm, and the distance between center axle of adjacent conical microneedles is 1 mm; and

the conical microneedle array diagram prepared in embodiment 4 is shown in FIG. 3c of FIG. 3.

### Embodiment 5

Embodiment 5 is almost the same as Embodiment 1, and the difference is only that:

in the step (2), a conical microneedle master mold at the aspect ratio of 3:1 is obtained by the MDX-50 desktop type automatic milling machine. The height of the conical microneedle is 0.9 mm, the bottom diameter is 0.3 mm, and the distance between center axle of adjacent conical microneedles in a longitudinal or horizontal direction is 1 mm; and

the conical microneedle array diagram prepared in embodiment 5 is shown in FIG. 3d.

### Embodiment 6

Embodiment 6 is almost the same as Embodiment 1, and the difference is only that: a conical microneedle master mold at the aspect ratio of 3:1 is obtained by the MDX-50 desktop type automatic milling machine. The height of the conical microneedle is 0.9 mm, the bottom diameter is 0.3 mm, and the distance between center axle of adjacent conical microneedles is 1 mm. The large array contains 35×35 arrays, and small array contains 6×6 arrays.

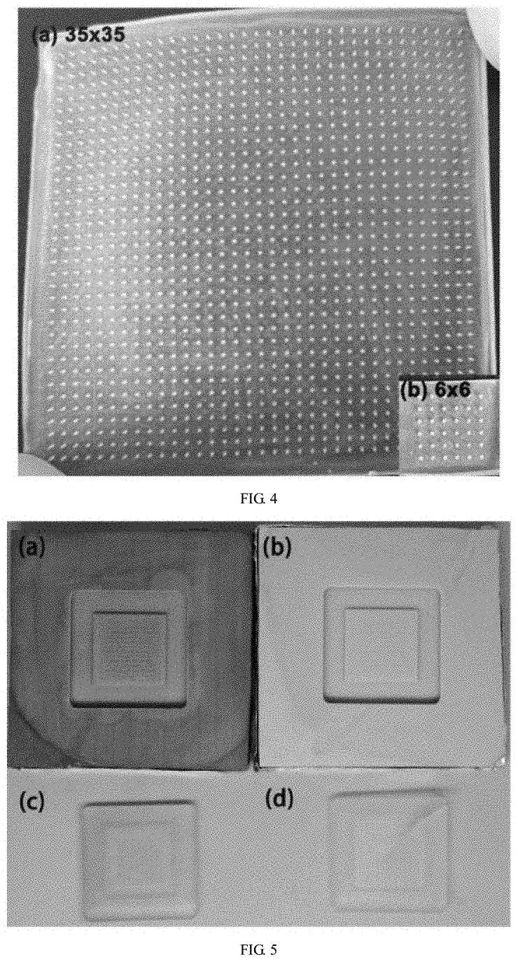

The array diagram of the conical microneedle on the microneedle patch prepared in embodiment 6 is shown in FIG. 4.

### Embodiment 7

Embodiment 7 is almost the same as Embodiment 1, and the difference is only that:

in the step (3), the following method is adopted:

the surface oxygen plasma treatment is conducted on a surface of the master mold by the Plasma treatment instrument (Germany Diener, Femto), the treatment time is 15 min and the power is 70 W.

### Reference example 1

Reference example 1 is almost the same as Embodiment 1, and the difference is only that:

the mixture (Dow Corning SYLGARD 184, and the proportion of PDMS to curing agent is 10:1) containing the dimethyl silicone (PDMS) and the curing agent is casted on a surface of the master mold directly obtained in the step (2) without any treatment. After the bubbles from the mixture are removed by negative pressure, the master mold is placed in an oven for 1 h at 90° C. for curing, and PDMS daughter mold is obtained by demolding.

The results of step (4) of embodiment 1 and the results of PDMS daughter mold obtained by demolding in reference example 1 are shown in FIG. 5. It can be seen from FIG. 5 that in the step (4) of embodiment 1, the complete PDMS daughter mold can be reversed, and the demolding is easy. However, in reference example 1, it is difficult to demold the PDMS daughter mold from the untreated master mold, and the daughter mold is easy to be broken, which is not conducive to the subsequent preparation for microneedle patches.

All references to the present invention are cited as references in the present application, just as each reference is cited as a reference separately. In addition, it should be understood after reading the above content of the present invention, those skills in the art could implement various modifications. Variations of the present invention and such equivalent forms also fall within the scope defined by appended claims of the present application.

1. A preparation method for a microneedle patch, wherein the method comprises the steps of:
(1) milling a base into a master mold of the microneedle patch;
(2) conducting surface treatment on the master mold, to obtain the treated master mold;
(3) conducting reverse molding on a surface of the treated master mold, conducting deaeration, curing, and demolding, to obtain the cured daughter mold; and
(4) casting the modification solution on the surface of the daughter mold, and then conducting deaeration, drying and curing, to obtain the microneedle patch;
in the step (1), the milling is carried out by the milling machine, and the milling machine is an MDX-50 desktop type automatic milling machine;
in the step (1), the aspect ratio of the microneedles is (1-3):1;
in the step (2), the surface treatment is gold-sputtering treatment, and the sputtering treatment conditions are: the current is 5-20 mA, and the sputtering time is 0.5-1.5 min;
in the step (3), the reverse molding is conducted on the surface of the treated master mold by the mixture containing dimethyl silicone and curing agent;
the mixture containing the dimethyl silicone and the curing agent is from Dow Corning SYLGARD 184 kits;
the weight ratio of the dimethyl silicone to curing agent is 8-12:1; and
the modification solution is PVA aqueous solution with the weight fraction of 2%-20%.

2. The method of claim 1, wherein the modification solution is PVA aqueous solution with the weight fraction of 6%-l0wt%.

3. The method of claim 1, wherein the current is 10-18 mA.

4. A microneedle patch, wherein the microneedle patch is prepared by the method of claim 1.
